(19) United States
(12) Patent Application Publication
(10) Pub. No.: US 2002/0192173 A1
(43) Pub. Date: Dec. 19, 2002

(54) Anhydrous treatment compositions for the delivery of reactive agents to amino-acid based substrates

(75) Inventors: Robert Wayne Glenn JR., Surrey (GB); Peter Marte Torgerson, Washington Courthouse, OH (US)

Correspondence Address: THE PROCTER & GAMBLE COMPANY, PATENT DIVISION, SHARON WOODS TECHINICAL CENTER, 11511 REED HARTMAN HIGHWAY, CINCINNATI, OH, 45241, US

(73) Assignee: The Procter & Gamble Company

(21) Appl. No.: 10/051,401

(22) Filed: Jan. 18, 2002

Related U.S. Application Data
(60) Provisional application No. 60/263,303, filed on Jan. 22, 2001.

(51) Int. Cl. A61K 7/06

(52) U.S. Cl. 424/70.1

(57) ABSTRACT

Disclosed are anhydrous treatment compositions comprising a reactive agent and a solvent in which the reactive agent is soluble and which is water-miscible. Also disclosed are compositions comprising greater than about 25% water-miscible solvent, from about 0.5% to about 25% surfactant, and water wherein the composition is thickened by high solvent containing lamellar liquid crystals.

### CROSS REFERENCE TO RELATED APPLICATION

This application claims the benefit of U.S. Provisional Application No. 60/263,303 filed on Jan. 22, 2001.

### TECHNICAL FIELD

The present invention relates to compositions to be used to treat amino-acid based substrates with reactive cosmetic actives, such as reactive conditioners, dyes, styling aids, sunscreens, etc. The compositions are chemically shelf stable formulations and are aesthetically acceptable to consumers. The present invention also relates to systems for treating amino-acid based substrates comprising the anhydrous compositions containing the actives and a separately packaged aqueous composition. Further, the present invention also relates to the methods of treating amino-acid based substrates wherein the anhydrous composition is applied to the substrate either alone or as part of the two-component system.

### BACKGROUND OF THE INVENTION

Consumers have been treating amino acid based substrates for years. Such treatments have included the waterproofing or coloring of textiles, the sunscreening of skin, the coloring, conditioning, and styling of hair, the dentifrice treatment of teeth, and more. It is well known that if such treatments can be done by safe covalent attachment to the substrate, the treatment will be much more long lasting. Therefore, several reactive chemistries have been developed to provide covalent attachment to amino acid based substrates, such as hair. Historically, these technologies, based on covalent attachment of cosmetic actives, have primarily relied upon electrophilic (electron accepting) and nucleophilic (electron donating) reactive groups or “hooks”. (Please see WO98/38974 by G. Malle et. al., U.S. Pat. No. 5,935,560 by J. Seper et. al., U.S. Pat. No. 5,525,332 by A. Gough et. al., WO96/03966 by P. Baile et. al., U.S. Pat. No. 5,523,080 by A. Gough et. al., WO94/26237 by A. Gough et. al., U.S. Pat. No. 5,350,572 by A. Savaides et. al., U.S. Pat. No. 5,254,335 by T. Deppert et. al., U.S. Pat. No. 5,206,013 by T. Deppert et. al., U.S. Pat. No. 5,211,942 by T. Deppert et. al., EP0 437099 A1 by D. Halloran et. al., CA 2024509AA by B. Murphy et. al., U.S. Pat. No. 5,087,733 by T. Deppert et. al., U.S. Pat. No. 5,030,756 by T. Deppert et. al., U.S. Pat. No. 4,973,475 by R. Schnetzinger et. al., GB 2197887A by C. Mahieu et. al., U.S. Pat. No. 4,567,039 by R. Stadnick et. al., EPO 159628 by R. Stadnick et. al., U.S. Pat. No. 4,102,641 by A. Cunningham et. al., U.S. Pat. No. 3,549,602 by G. Kalopissis et. al., U.S. Pat. No. 3,484,417 by G. Kalopissis et. al.) More recently, a protected thiol “hook” technology for the covalent attachment of cosmetic actives to amino acid substrates has been proposed. (Please reference WO2000/040210 A2 by R. Glenn et. al., and WO2000/040209 A2 by R. Glenn.)

Whereas these reactive groups or “hook” moieties are reactive towards amino acid residues in proteins, they are also reactive towards electron rich ingredients that are employed in the formulation of typical delivery systems, such as aqueous based formulations and other electron rich compounds such as atmospheric oxygen. This leads to pre-mature decomposition of the hooks of the covalent reactive compounds, referred to herein as the reactive agents, over the shelf life of the product which severely or completely mitigates reactive efficacy with a substrate when used by the consumer. As would be expected, delivery of reactive agents in anhydrous solvents resolves the stability issues. However, typical anhydrous solvents entrap the active in the anhydrous phase preventing transport of the active to the substrate which is to be treated. It has been discovered that when the anhydrous solvent is specifically selected, the resulting anhydrous composition can be made to be shelf stable and sufficiently reactive in the presence of water to covalently treat the substrate. The anhydrous solvent is chosen such that the active is soluble in the solvent and the solvent is at least partially miscible in water.

Preferred methods of using the anhydrous compositions of the present invention include the application of the compositions to substrates which have been pre-moistened with water or application to the substrate after mixing the anhydrous composition with water immediately before use.

A preferred embodiment of the present invention is use of the anhydrous compositions in a system comprising the anhydrous composition containing the reactive agent within a water-miscible solvent and a separately packaged aqueous composition. The separately packaged compositions of the system are mixed by the consumer, just prior to or simultaneous to use. Accordingly, the reactive agent is protected from moisture and oxygen in the environment via inclusion within an anhydrous solvent during production, packaging, shipment and storage, and yet may be applied to hair in a consumer desired product form with a significant quantity of solvent and without sacrificing hair feel.

Especially preferred systems form a solvent/aqueous solution that is thickened via liquid crystals in the presence of a high solvent level. Additionally, specialized surfactants may be included in either composition such that upon intermixing of the two compositions by the consumer, either immediately prior to or simultaneous to use, a thickened/non-drip solution is formed that is comprised of lamellar liquid crystals that are robust enough to withstand a high solvent load while still providing viscosity build. The intermixing of the two phases can be accomplished via a number of packaging means.

Achieving viscosity build under a high solvent load (i.e., >50%) is a challenge. Most conventional thickeners, such as hydroxyethyl cellulose, xanthan gum, Crothix®, Thixcin R®, fumed silica, hydrophobically modified fumed silica, cetylhydroxyethyl cellulose, bentonite clays, natural waxes, guar gums, etc., fail to produce significant viscosity build at levels up to 5% by weight. As reported in the literature for high alcohol containing systems, e.g., antiperspirants (EP792635 A2 by I. Esser and S. Jones), deodorants (EP674899 A2 by M. Correia et. Al.) and hand disinfectants (JP11349418 A2 by K. Yokota et. Al.), hydroxypropyl cellulose and hydroxypropyl methyl cellulose are used to provide viscosity build. Indeed, both of these polymers provide acceptable viscosity build even at levels as low as 1%. However, whereas these polymers are acceptable for skin care applications, they have been found to leave a highly undesirable ‘sticky’ film on hair, making them unacceptable as a solvent thickener for hair applications.

Surprisingly, it has been discovered that the heavily solvent laden solution can be thickened via liquid crystals even under very low water levels as low as 10-40%. This is in contrast to the conventional use of liquid crystals which spans exclusively high water containing/aqueous systems, i.e., aqueous hair conditioners, shower gels, body lotions etc. While it has been determined that liquid crystals have difficulty maintaining viscosity in the presence of the high solvent load over long time periods, i.e., weeks/months, they have been found to produce robust thickening for a time period in far excess of the time required for application of the treatment solution to hair after intermixing both phases. Generally, an acceptable viscosity build of the solvent solution lasts for several days which is well beyond the 5 to 30 minutes required for treatment to the hair.

Importantly, the high solvent containing liquid crystals of the present invention have significant advantages over the conventional use of hydroxypropylcellulose as a solvent thickener for hair applications. First, the liquid crystals, being themselves small molecules, are more easily rinsed away from the hair and do not leave the hair in a ‘sticky’ or ‘tacky’ state after treatment. Second, the liquid crystals produce a ‘moundy’ rheology with a significant yield point which differs from the ‘stringy’/‘gooey’ rheology produced by the likes of hydroxypropylcellulose. Third, the liquid crystals are comprised of amphiphilic molecules that produce a consumer desired conditioning effect to the hair.

### SUMMARY OF THE INVENTION

Disclosed are anhydrous treatment compositions comprising a reactive agent and a solvent in which the reactive agent is soluble and which is water-miscible. Also disclosed are compositions comprising greater than about 25% water-miscible solvent, from about 0.5% to about 25% surfactant, and water wherein the composition is thickened by high solvent containing lamellar liquid crystals.

### DETAILED DESCRIPTION OF THE INVENTION

The present invention relates to delivery of reactive agents via anhydrous compositions comprising specially selected water-miscible solvents which result in chemically stable compositions which are sufficiently reactive to treat amino-acid based substrates in a consumer acceptable method.

The term “amino acid based substrates”, as used herein, refers to proteinaceous materials, such as keratin, as found in human and animal hair, skin, and nails. Amino acid based substrates useful herein are hydroxyl-containing, amine-containing, thiol-containing, and disulfite-containing amino acids.

The term “covalently reactive”, as used herein, refers to the ability of reactive agents to form covalent bonds with functional groups within proteinaceous keratin, e.g., with keratin amino acids comprising —SH, —OH, —NH₂ or —S—S— groups, thereby forming a permanent bond with the keratin that is resistant to shampooing or cleansing.

The term “water-miscible solvent”, as used herein, refers to solvents which can solubilize the reactive agent, and which are at least partially miscible with water, meaning that the solvent will remain a single-phase system while mixed with up to 10% water by weight.

The term “reactive agent”, as used herein, refers to compounds that comprise a reactive group or “hook” that is covalently reactive with keratin and a mono or multivalent cosmetically active functional group that imparts one or more visual, tactile or other cosmetic beneficial effects on proteinaceous materials such as keratin, hair, skin, animal fur or wool.

The term “chemically shelf stable” or “chemically stable”, as used herein, applies to a composition comprising a reactive agent wherein the reactive agent does not chemically decompose substantially (via hydrolysis, reduction or oxidation) over the desired shelf life of the product such that the reactive agent maintains its ability to react with the proteinaceous substrate.

The term “anhydrous”, as used herein, refers to compositions which are essentially free of water. By essentially free, it is meant that the anhydrous composition contains not more than 0.1%.

The treatment compositions of the present invention can comprise, consist of, or consist essentially of the essential elements and limitations of the invention described herein, as well as any of the additional or optional ingredients, components, or limitations described herein.

All percentages, parts and ratios are by weight of the total composition, unless otherwise specified. All such weights as they pertain to listed ingredients are based on the specific ingredient level and, therefore, do not include solvents, carriers, by-products, filler or other minor ingredients that may be included in commercially available materials, unless otherwise specified.

Anhydrous Treatment Composition

The anhydrous treatment composition of the present invention comprises a chemically unstable reactive agent and a water immiscible solvent which solubilizes the reactive agent. The reactive agent comprises from about 0.01% to about 10%, more preferably from about 0.25% to about 5%, and most preferably from about 0.5% to about 2% by weight of the composition. The water-miscible solvent comprises from about 25% to about 95%, more preferably from about 30% to about 90%, and most preferably from about 50% to about 85% by weight of the composition.

The anhydrous composition generally comprises the reactive agent solubilized or dispersed within the water-miscible solvent. Optional ingredients can also be incorporated including perfume, surfactants, preservatives, humectants, emollients, conditioners, stabilizers and the like. The anhydrous composition of the present invention can be formulated as a fluid, a serum, or a gel having a viscosity from 1 to 100,000 mPas or above.

The anhydrous composition is essentially free of water and should be stored in such a way that minimizes contact or exposure to the atmosphere.

Reactive Agent

The reactive agent comprises a reactive group or “hook” and a mono or multivalent cosmetically active functional group that imparts one or more visual, tactile or other cosmetic beneficial effects on proteinaceous materials such as keratin, hair, skin, animal fur or wool. The reactive agent comprises one or more reactive groups selected from the group consisting of electrophilic, nucleophilic, protected thiol groups and mixtures thereof.

Disclosed technologies for the covalent attachment of cosmetic actives (primarily dyes and conditioners) to hair keratin have primarily relied upon electrophilic (electron accepting) and nucleophilic (electron donating) reactive groups or “hooks”. Please reference WO98/38974 by G. Malle et. al., U.S. Pat. No. 5,935,560 by J. Seper et. al., U.S. Pat. No. 5,525,332 by A. Gough et. al., U.S. Pat. No. 5,656,265 by P. Baile et. al., U.S. Pat. No. 5,523,080 by A. Gough et. al., WO94/26237 by A. Gough et. al., U.S. Pat. No. 5,350,572 by A. Savaides et. al., U.S. Pat. No. 5,254,335 by T. Deppert et. al., U.S. Pat. No. 5,206,013 by T. Deppert et. al., U.S. Pat. No. 5,211,942 by T. Deppert et. al., EPO 437099 by D. Halloran et. al., CA 2024509AA by B. Murphy et. al., U.S. Pat. No. 5,087,733 by T. Deppert et. al., U.S. Pat. No. 5,030,756 by T. Deppert et. al., U.S. Pat. No. 4,973,475 by R. Schnetzinger et. al., GB 2197887A by C. Mahieu et. al., U.S. Pat. No. 4,567,039 by R. Stadnick et. al., EPO 159628 by R. Stadnick et. al., U.S. Pat. No. 4,102,641 by A. Cunningham et. al., U.S. Pat. No. 3,549,602 by G. Kalopissis et. al., U.S. Pat. No. 3,484,417 by G. Kalopissis et. al. Also disclosed are protected thiols reactive “hooks” for the covalent modification of hair keratin. Please reference U.S. patent applications having the Ser. Nos. 09/478,855 and 09/227,912, both by R. Glenn et al.

Electrophilic reactive groups or “hooks” that may be included within reactive agents of the present inventions include, but are not limited to, the following: Azolactones as described in U.S. Pat. No. 5,656,265 by P. Bailey et. al., and U.S. Pat. Nos. 5,523,080 and 5,525,332 both by A. Gough et. al.; Alkyl halides as described in U.S. Pat. Nos. 5,211,942 and 5,030,756 by T. Deppert et. al.; Dithiocarboxylic acid esters as described in DE2,310,420 by N. Leon et. al.; Thiosulfates as described in U.S. Pat. No. 3,415,606 by R. Randebrock and Seifen-Oele-Fette-Wachse 89:147 (1963) by G. T. Walker; Acyl halides as described in GB2,197,887 by C. Mahieu et. al.; Acrylates as described in the J. Soc. Dyers Col, 70:112 (1954) by G. W. Madaras and J. B. Speakman; Maleimides as described in Textile Research Journal, August 1957, pages 615-622 by R. Burley and F.W.A. Horden; Halotriazines and halopyrimidines as described in GB951,021 assigned to Turner Hall Corporation and U.S. Pat. No. 3,340,000 by A. Shansky; Vinylsulfones as described in GB951,021 assigned to Turner Hall Corporation; Urea derivatives as described in U.S. Pat. No. 3,725,525 by B. Joos; Alkoxysilanes as described in EP0159628 by R. Stadnick and U.S. Pat. No. 4,567,039 by R. Stadnick et. al.; Isothiuroniums as described in U.S. Pat. Nos. 5,254,335 and 5,206,013 both by T. Deppert et. al.; and Haloquinoxalines, β-haloethylsulfones, β-sulfatoethylsulfones, acrylates, methacrylates, acrylamides, methacrylamides, epoxides, esters, carbamates, anhydrides, isothiocyanates, isocyanates, and lactones as described in WO98/38974 by G. Malle et. al. These references are all incorporated by reference herein.

Nucleophilic reactive groups or “hooks” that may be included within reactive agents of the present inventions include, but are not limited to, the following: thiols or thiolates as described in U.S. Pat. No. 3,484,417 by G. Kalopissis et. al., U.S. Pat. Nos. 5,935,560 and 5,935,560, both by J. Seper et. al., U.S. Pat. No. 5,776,454 by R. Gee et. al.; thiols or thiolates containing quaternary salts as described in U.S. Pat. No. 4,973,475 by R. Schnetzinger et. al., U.S. Pat. Nos. 5,087,733 and 5,206,013 both by T. Deppert et. al.; thioalkylamides as described in EP0437099 by D. Halloran et. al.; thioalkyl esters as described in U.S. Pat. No. 5,350,572 by A. Savaides et. al., and cysteamine derivatives as described in Textile Res. J. 67(7), 486-492 (1997). These references are all incorporated by reference herein.

Protected thiol reactive groups or “hooks” that may be included within reactive agents of the present inventions include, but are not limited to, reactive groups of the following structure:

R—(S—Pr)ₘ

where R is a mono or multivalent cosmetically active functional group, S is sulfur, Pr is a protecting group and m is an integer between 1 and 100. The protecting group is selected from the group consisting of heterocyclic protecting groups, Sp aliphatic trigonal carbon protecting groups, sp³ carbon electrophilic protecting groups, phosphorus protecting groups, metal based protecting groups, non-metal and metalloid based protecting groups, energy-sensitive protecting groups and mixtures thereof as described in U.S. patent applications having the Ser. No. 09/478,866 and 09/227,912, both by R. Glenn et al., and both of which are incorporated by reference herein.

Preferred reactive groups or “hooks” of the present invention include those of the protected thiol type. Of the thiol protective groups, the heterocyclic protecting groups, the sp² protecting groups and the phosphorus protecting groups are preferred, with the heterocyclic protecting groups being more preferred. Of the heterocyclic protecting groups, the pyrimidinium, pyridinium, and benzothiazolium classes are preferred, with the pyrimidinium class being more preferred.

The mono or multivalent cosmetically active functional group, R, suitable for inclusion within the reactive agents of the present invention may be any moiety that imparts one or more visual, tactile or other cosmetic beneficial effects on proteinaceous materials such as keratin, hair, skin, animal fur or wool. Any cosmetic moiety may be included as a functional group in the compositions of the present invention as long as the compound can be modified to contain at least one reactive group or “hook” as described herein and in the references provided herein.

Suitable functional groups that are suitable for inclusion within the reactive agents of the present invention include, but are not limited to, antimicrobial compounds, UV-absorbing compounds, skin conditioning agents, hair conditioning agents, hair repair agents, hair styling agents, hair dyes, scalp treatment agents, anti-inflammatory compounds, antioxidants, dyes and coloring agents, perfumes, oral care actives, skin moisturizers, pharmaceutical agents, antidandruff agents, insect repellents, moisturizers, humectants, pearlescent and/or opacifying materials, fabric care actives, pet grooming actives, fabric anti-wrinkling agents, shrink-resistant actives, laundry care actives, hard surfaces actives, textile actives, textile dyes, water-proofing agents, cationic polymers, cationic surface modifiers, hydrophobic surface modifiers, anionic surface modifiers, absorbents, antifungal agents, insecticidal agents, textile color guards, nail actives such as enamel and polish, eyelash actives and mascara, antiperspirant and deodorant actives, anti-acne actives, odor control actives, fluorescent actives, bleaching agents, enzymes, antibodies, dispersing aids, emollients, stabilizers, anti-static agents, anti-seborrhea agents, optical brighteners, fluorescent dyes, softeners, cross-linking agents, photobleaches, bactericides, and mixtures thereof. Please see U.S. Ser. No. 09/478,855 by R. Glenn et. al. for a more detailed listing of cosmetic functional groups.

Preferred cosmetic functional groups include hair conditioning agents, hair repair agents, hair styling agents, and hair dyes and coloring agents. Please see U.S. Ser. No. 09/478,855 by R. Glenn et. al. for a more thorough list of preferred cosmetic functional groups.

An exemplary reactive agent to demonstrate the present invention comprises a Protected Thiol reactive group of the pyrimidinium type combined with a silicone hair conditioning cosmetic functional group. The structure of this exemplary reactive agent is as follows:

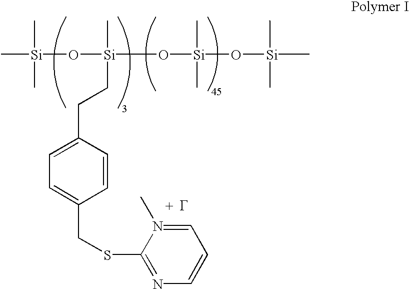

This compound and its synthesis preparation are disclosed within U.S. application Ser. No. 09/478,855 by R. Glenn et. al. which is incorporated by reference herein. Additional reactive agents containing a silicone hair conditioning cosmetic functional group are be found within U.S. application Ser. No. 09/616,535 by M. Butts et al., filed Jul. 14, 2000, U.S. application Ser. No. 09/616,534 by M. Butts et al., filed Jul. 14, 2000, U.S. application Ser. No. 09/616,533 by M. Butts et al., filed Jul. 14, 2000, U.S. application Ser. No. 09/616,532 by M. Butts et al., filed Jul. 14, 2000, all of which are incorporated by reference herein. Polymer I is chemically shelf unstable with the pyrimidinium moiety being prone to premature hydrolysis in the presence of aqueous media.

An additional exemplary reactive agent to demonstrate the present invention comprises an electrophilic reactive group of the azlactone type combined with a silicone hair conditioning cosmetic functional group. The structure of this exemplary reactive agent is as follows:

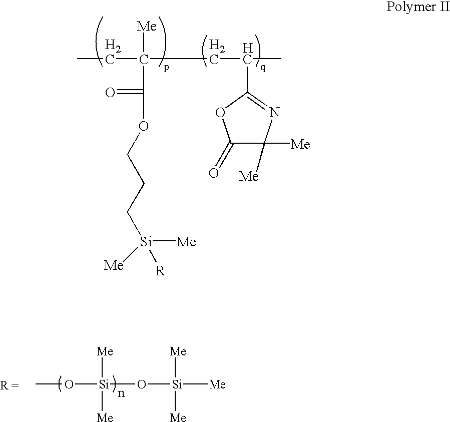

where p=1, q=14.6 and n=60. This compound and its synthesis preparation is disclosed within U.S. Ser. No. 5,525,332 by A. D. Gough et. al. from column 12, lines 11-67 through Column 13, lines 1-18.

An additional exemplary reactive agent to demonstrate the present invention comprises a nucleophilic reactive group of the thiol type combined with a hydrocarbon conditioning cosmetic functional group. The structure of this exemplary reactive agent is as follows:

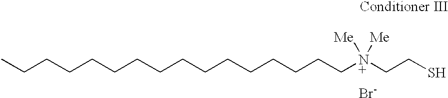

This compound and its synthesis preparation is disclosed within U.S. Ser. No. 5,087,733 by T. M. Deppert et. al. from column 6, lines 35-68 and Column 7, lines 10-15, which is herein incorporated by reference.

Water-Miscible Solvent

The water-miscible solvent of the present invention comprises one or more solvents that must be able to dissolve reactive agent while being at least partially miscible with water. The water-miscible solvents include solvents in which the reactive agent is not fully dissolved in the solvent, but the partial solution or dispersion is such that it is physically stable. That is, the solution/dispersion does not settle out, or separate into more than one phase, over time. Such solutions or dispersions are often clear, but may be turbid or cloudy.

Suitable solvents of the present invention include, but are not limited to, amides, esters, ethers, ketones, cyclic amides, cyclic esters, cyclic ketones, and cyclic ethers having a Vaughan Solubility Parameter ranging from about 8.0 (cal/cm³)^{0.5} to about 17.0 (cal/cm³)^{0.5}. Solubility parameters for the solvents, and means for determining such parameters, are well known in the chemical arts. More specifically, the Vaughan Solubility Parameter (VSP) (δ) is a calculated parameter used to define an material's solubility and is defined as the sum of all the cohesive forces and the square root of the energy of vaporization. The VSP typically has a range of 5-25. A further description of solubility parameters and means for determining them are described by C. D. Vaughan, “Solubility Effects in Product, Package, Penetration and Preservation” 103 Cosmetics and Toiletries 47-69, October 1988; and C. D. Vaughn, “Using Solubility Parameters in Cosmetics Formulation”, 36 J. Soc. Cosmetic Chemists 319-333, September/October, 1988, which descriptions are incorporated herein by reference.

Nonlimiting examples of suitable solvents for use within the present invention include ethyl formate, dimethyl isosorbide, acetylacetone, 2-butanone, acetone, methyl acetate, ethyl acetate, propyl acetate, ethoxyethanol, dipropylene glycol monomethyl ether, butyl lactate, t-butyl alcohol, phenyl acetate, 2-propoxyethanol, 2-isopropoxyethanol, methoxypropanol, isopropyl lactate, hexyl alcohol, butoxyethanol, tripropylene glycol (PPG-3), triacetin, methoxyethanol, isopropyl alcohol, PEG-8, methyl lactate, PEG-6, PEG-5, PEG-4, N-methylpyrrolidone, propyl alcohol, dipropylene glycol (PPG-2), acetonitrile, phenoxyethanol, triethylene glycol, hexylene glycol, ethyl alcohol, γ-butyrolactone, butylene glycol, propylene carbonate, dimethyl sulfoxide, diethylene glycol, ethoxydiglycol, propylene glycol, pyrrolidone, pyrrolidone-2, methyl alcohol, ethylene carbonate, ethylene glycol, acetamide, glycerin, butyl carbitol, 1,3-dioxolane, dimethoxymethane, 1,2-hexanediol, dipropylene glycol butyl ether, dipropylene glycol t-butyl ether, propionaldehyde, diethoxymethane, glycerol formal, γ-valerolactone, α-methyl-γ-butyrolactone, and butyl acetate and mixtures thereof.

The VSP's for some of these solvents are set forth in Table I:

TABLE I

| Solvent | Vaughn Solubility Parameter |
|---|---|
| Butyl acetate | 8.93 |
| Ethyl formate | 9.55 |
| Dimethyl isosorbide | 9.58 |
| Acetylacetone | 9.68 |
| 2-butanone | 9.8 |
| Acetone | 9.87 |
| Methyl acetate | 9.88 |
| Ethoxyethanol | 9.9 |
| Dipropylene glycol monomethy | 9.99 |
| Butyl lactate | 10.27 |
| t-butyl alcohol | 10.28 |
| Phenyl acetate | 10.33 |
| Methoxypropanol | 10.4 |
| Isopropyl lactate | 10.42 |
| Hexyl alcohol | 10.5 |
| Butoxyethanol | 10.53 |
| Tripropylene glycol (PPG-3) | 10.6 |
| Tracetin | 10.77 |
| Methoxyethanol | 10.8 |
| Isopropyl alcohol | 11.24 |
| PEG-8 | 11.34 |
| Methyl lactate | 11.47 |
| PEG-6 | 11.47 |
| PEG-5 | 11.57 |
| PEG-4 | 11.61 |
| N-methylpyrrolidone | 11.71 |
| Propyl alcohol | 11.73 |
| Dipropylene glycol (PPG-2) | 11.78 |
| Acetonitrile | 11.81 |
| Phenoxyethanol | 11.87 |
| Triethylene glycol | 12.21 |
| Hexylene glycol | 12.32 |
| Ethyl alcohol | 12.55 |
| Gamma-butyrolactone | 12.85 |
| Butylene glycol | 13.2 |
| Propylene carbonate | 13.35 |
| Dimethyl sulfoxide | 13.4 |
| Diethylene glycol | 13.61 |
| Ethoxydiglycol | 13.8 |
| Propylene glycol | 14 |
| Pyrrolidone | 14 |
| Pyrrolidone-2 | 14.22 |
| Methyl alcohol | 14.33 |
| Ethylene carbonate | 14.45 |
| Ethylene glycol | 14.5 |
| Acetamide | 16.03 |
| Glycerin | 16.26 |

Preferred solvents of the present invention include, but are not limited to N-methyl-2-pyrrolidone, available commercially as M-PYROL (NMP) from ISP Europe, Tadworth, Surrey, England; γ-butyrolactone available commercially as BLO from ISP Europe, Tadworth, Surrey, England; γ-valerolactone available commercially as Gamma Valerolacdtone C-5 from Soda Aromatic Co., LTD, Tokyo, Japan; propylene glycol available commercially as 1,2-Propylene Glycol USP from BASF Corporation or Eastman Propylene Glycol from Eastman Chemical Company; dipropylene glycol available commercially as Eastman Dipropylene Glycol from Eastman Chemical Company; ethoxydiglycol available commercially as Eastman DE Solvent from Eastman Chemical Company; ethoxyethanol available commercially as CELLOSOLVE Solvent from Union Carbide Corporation; dimethoxymethane available commercially as Methylal from Alcohols Limited, Hertfordshire, England; dimethyl isosorbide available commercially as Arlasolve DMI from Uniqema, Everberg, Belgium; butyl lactate available commercially as CPS Butyl Lactate from CPS Chemical Company, Memphis, Tennessee; and mixtures thereof.

When solvent mixtures are employed, a preferred solvent for inclusion includes propylene carbonate provided that the resultant solvent mixture is miscible with water. Propylene carbonate is available commercially as ARCONATE PROPYLENE CARBONATE, available from ARCO Chemical Company.

Ethyl alcohol and isopropyl alcohol are also preferred solvents in cases where they do not adversely affect the chemical stability of the reactive agent. In general, however, chemical stability is favored via use of more lowly protic and aprotic solvent choices.

Treatment System

A preferred embodiment of the anhydrous treatment composition of the present invention includes a treatment system which comprises the anhydrous composition and a separately packaged aqueous composition. The treatment system comprises from about 50% to about 95%, preferably from about 55% to about 90% and more preferably from about 60% to about 80% of the anhydrous treatment composition and from about 5% to about 50%, preferably from about 10% to about 45%, more preferably from about 20% to about 40%, by weight of the total system, of the aqueous composition.

Aqueous Composition

The aqueous composition of the treatment system of the present invention may comprise water and optional components including surfactants, perfumes, preservatives, humectants, emollients, conditioners, stabilizers and the like. It is often desirable to include a pH buffer within the aqueous composition to control the aqueous pH upon application to hair which may facilitate the performance of the reactive agent. Numerous pH buffers are available to those skilled in the art including. For use, the composition may be provided at a pH from about 3 to 11, preferably from 4 to 10 that may or may not be pH buffered. The aqueous composition of the present invention can be formulated as a fluid, lotion, fluid cream or cream having a viscosity from 500 to 100,000 mPas or above. The aqueous composition is package separately from the anhydrous composition.

Optional Ingredients

Numerous optional ingredients may be included in either one or both of the respective anhydrous and aqueous compositions of the present invention in so far as the optional ingredients or adjuncts do not eliminate or substantially reduce the performance of the reactive agent upon intermixing the compartments together and applying the resulting solution to the hair. The additional ingredients may include, for example dyes and coloring agents, fragrances; anionic, cationic, non-ionic, amphoteric or zwitterionic surfactants; buffers, masking fragrances, dispersing agents, stabilizers, cationic polymers, perfumes, non-ionic polymers, anionic polymers, complex coacervates, complex coacervate capsules, metal salts, lewis acids, buffering agents, particulate thickeners, polymeric thickeners, wax thickeners, oils, emollients, humectants, moisturizers, dyes, dyes and coloring agents, pearlescents, opacifiers, enzymes, suspending agents, antimicrobials, preservatives, proteins, herb and plant extracts, bleach, peroxide, polyols, silicones, oils, antibodies, pH adjusting agents including pH buffers, viscosity modifiers, preservatives, viscosity enhancers, gelling agents, chelators, silicones, emulsifying agents, moisturizing and conditioning agents, and other common adjuvants well known to those skilled in the art.

An antioxidant may also be incorporated within the composition. Suitable antioxidants include vitamin E and its derivatives, BHT and BHA.

The aqueous composition of the present invention may optionally contain from about 0.1% to about 10%, preferably from about 0.25% to about 8%, more preferably from about 0.5% to about 5% of a stabilizer. The stabilizer is used to form a stabilizing network within the bi-layer emulsion that prevents phase separation.

In one embodiment of the present invention, the stabilizer can comprise a crystalline, hydroxyl-containing stabilizer. The crystalline, hydroxy-containing stabilizer is selected from the group consisting of:

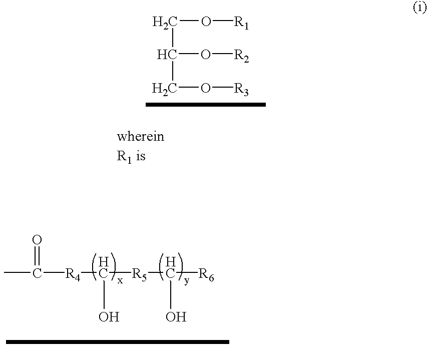

R₂ is R₁ or H

R₃ is R₁ or H

R₄ is C₀₋₂₀ Alkyl

R₅ is C₀₋₂₀ Alkyl

R₆ is C₀₋₂₀ Alkyl

R₄+R₅+R₆=C₀₋₂₂

and wherein 1≦x+y≦4;

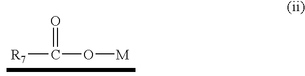

wherein R₇ is —R₄(CHOH)ₓR₅(CHOH)_{y}R₆

M is Na⁺, K⁺ or Mg₊₊, or H; and

mixtures thereof;

Some preferred hydroxyl-containing stabilizers include 12-hydroxystearic acid, 9,10-dihydroxystearic acid, tri-9,10-dihydroxystearin and tri-12-hydroxystearin (hydrogenated castor oil is mostly tri-12-hydroxystearin). Tri-12-hydroxystearin is most preferred for use in the emulsion compositions herein.

When these crystalline, hydroxyl-containing stabilizers are utilized in the personal cleansing compositions herein, they are typically present at from about 0.1% to 10%, preferably from 0.2% to 5%, more preferably from 0.4% to about 2% of the treatment compositions. The stabilizer is insoluble in water under ambient to near ambient conditions.

Alternatively, the stabilizer employed can comprise a polymeric thickener. When polymeric thickeners as the stabilizer in the personal cleansing compositions herein, they are typically included in an amount ranging from about 0.01% to about 5%, preferably from about 0.3% to about 3%, by weight of the composition. The polymeric thickener is preferably an anionic, nonionic, cationic or hydrophobically modifier polymer selected from the group consisting of cationic polysaccharides of the cationic guar gum class with molecular weights of 1,000 to 3,000,000, anionic cationic and nonionichomopolymers derived from acrylic and/or methacrylic acid, anionic cationic and nonionic cellulose resins, cationic copolymers of dimethyldialkylammonium chloride and acrylic acid, cationic homopolymers of dimethylalkyl-ammonium chloride, cationic polyalkylene and ethoxypolyalkylene imines, polyethylene glycol of molecular weight from 100,000 to 4,000,000, and mixtures thereof. Preferably, the polymer is selected from the group consisting of Sodium Polyacrylate, hydroxy ethyl Cellulose, Cetyl Hydroxy Ethyl Cellulose, and Polyquatemium 10.

The polymeric thickener is preferably and anionic, nonionic, cationic or hydrophobically modified polymer of natural, modified natural or synthetic origin from plants, microbials, animals or petroleum raw materials including karaya gum, tragacanth gum, gum arabic, gum ghatti, guar gum, locust bean gum, quince seed, psyllium seed, tamarind seed, carrageenan, alginates, agar, larch gum, pectins, starches, xanthan gum, dextran, casein, gelatin, keratin, shellac, cellulose derivatives, guar derivatives, acrylic acid polymers, polyacrylamides, and alkylene/alkylene oxide polymers. Preferred polymeric thickeners include guar gum, available commercially as SUPERCOL U, U NF, SUPERCOL GF, SUPERCOL G2S, and SUPERCOL G3 NF from Aqualon and JAGUAR GUM from Rhone-Poulenc; xanthan gum, available commercially as KELTROL CG, KELTROL CG F, KELTROL CG T, KELTROL CG TF, KELTROL CG 1000, KELTROL CG RD, KELTROL CG GM, KELTROL CG SF, from Calgon, and RHODICARE S, RHODICARE XC, RHODICARE H, AND RHODICARE D, from Rhone-Poulenc; hydroxyethylcellulose, available commercially as NATRASOL 210 types and NATRASOL 250 types from Aqualon; hydroxypropyl guar, available commercially as JAGUAR HP-8, JAGUAR HP-11, JAGUAR HP-60, and JAGUAR H-79 from Rhone-Poulenc. Additional specific polymeric thickeners that are suitable for the present invention are given in Rheological Properties of Cosmetics and Toiletries, edited by Dennis Laba, 1993, by Marcel Dekker, Inc. on pages 57 through 121 (ISBN 0-8247-9090-1).

Alternatively, the stabilizer employed can comprise C10-C22 ethylene glycol fatty acid esters. C10-C22 ethylene glycol fatty acid esters can also desirably be employed in combination with the polymeric thickeners hereinbefore described. The ester is preferably a diester, more preferably a C14-C18 diester, most preferably ethylene glycol distearate. When C10-C22 ethylene glycol fatty acid esters are utilized as the stabilizer in the personal cleansing compositions herein, they are typically present at from about 3% to about 10%, preferably from about 5% to about 8%, more preferably from about 6% to about 8% of the treatment compositions.

Another class of stabilizer which can be employed is dispersed amorphous silica. As used s herein the term “dispersed amorphous silica” refers to small, finely divided non-crystalline silica having a mean agglomerate particle size of less than about 100 microns.

Fumed silica, which is also known as arced silica, is produced by the vapor phase hydrolysis of silicon tetrachloride in a hydrogen oxygen flame. It is believed that the combustion process creates silicone dioxide molecules which condense to form particles. The particles collide, attach and sinter together. The result of this process is a three dimensional branched chain aggregate. Once the aggregate cools below the fusion point of silica, which is about 1710° C., further collisions result in mechanical entanglement of the chains to form agglomerates, precipitated silicas and silica gels are generally made in aqueous solution. See, Cabot Technical Data Pamphlet TD-100 entitled “CAB-O-SIL.RTM. Untreated Fumed Silica Properties and Functions”, October 1993, and Cabot Technical Dat Pamphlet TD-104 entitled “CAB-O-SIL.RTM. Fumed Silica in Cosmetic and Personal Care Products”, March 1992, both of which are herein incorporated by reference.

The fumed silica preferably has a mean agglomerate particle size ranging from about 0.1 microns to about 100 microns, preferably from about 1 micron to about 50 microns, and more preferably from about 10 microns to about 30 microns. The agglomerates are composed of aggregates which have a mean particle size ranging from about 0.01 microns to about 15 microns, preferably from about 0.05 microns to about 10 microns, more preferably from about 0.1 microns to about 5 microns and most preferably from about 0.2 microns to about 0.3 microns. The silica preferably has a surface area greater than 50 sq. m/gram, more preferably greater than about 130 sq. m./gram, most preferably greater than about 180 sq. m./gram.

When amorphous silicas are used as the stabilizer herein, they are typically included in the compositions at levels ranging from about 0.1% to about 10%, preferably from about 0.25% to about 8%, more preferably from about 0.5% to about 5%.

A fourth class of stabilizer which can be employed comprises dispersed smectite clay selected from the group consisting of bentonite and hectorite and mixtures thereof. Bentonite is a colloidal aluminum clay sulfate. See Merck Index, Eleventh Edition, 1989, entry 1062, p. 164, which is incorporated by reference. Hectorite is a clay containing sodium, magnesium, lithium, silicon, oxygen, hydrogen and fluorine. See Merck Index, eleventh Edition, 1989, entry 4538, p. 729, which is herein incorporated by reference. When smectite clay is employed as the stabilizer in the treatment compositions of the present invention, it is typically included in amounts ranging from about 0.1% to about 10%, preferably from about 0.25% to about 8%, more preferably from about 0.5% to about 5%.

Typically, a portion of the water-miscible solvent is included within the aqueous compartment to facilitate a more rapid viscosity build upon intermixing with the solvent. While not being bound to theory, it is believed that the liquid crystalline bi-layers formed within the aqueous compartment are initially disrupted upon intermixing with the anhydrous solvent, after which they reform in the predominantly solvent solution. By including a portion of the solvent within the aqueous compartment, it has been found that the initial viscosity disruption is minimized with the resulting viscosity build happening considerably faster, i.e., within seconds. The water-miscible solvent can be included within the aqueous compartment at levels from 1% to 70%, preferably from 20% to 60% and more preferably from 30% to 50% by weight of the aqueous composition.

In one embodiment, the optional ingredients may comprise conventional hair treatment chemicals including: hair bleaching agents including, but not limited to, hydrogen peroxide, sodium percarbonate, sodium perborate, magnesium perborate, magnesium dioxide, barium binoxide and combinations thereof; hair permanent wave agents including, but not limited to, thioglycolic acid, thiolactic acid, cysteine, thioglycerol, thioglycollic hydrazide, thioglycolamide, and glycerol monothioglycollate, salts of hydrogen sulfide, salts of hydrogen cyanide, trihydroxymethyl phosphine or its precursor, tetrahydroxymethyl phosphonium chloride, borohydride, dithionite, hydrosulfite, and sulfoxylate and combinations thereof; oxidative hair dyes including, but not limited to, p-phenylenediamine, toluene-2,5-diamine, 2-methoxy-p-phenylenediamine, 2-chloro-p-phenylenediamine, toluene-3,4-diamine,o-amino-phenol, p-aminophenol, resorcinol, 1-naphthol, pyrogallol, 4-chlororesorcinol, 4-methoxy-m-phenylenediamine, m-phenylenediamine, hydroquinone and mixtures thereof; semi-permanent hair dyes including, but not limited to, 2-nitro-p-phenylenediamine, 4-nitro-o-phenylenediamine, HC red No. 3, HC yellow No. 2, HC yellow No. 4, HC blue No. 1, HC red No. 1, HC orange No. 1, Disperse black 9, Acid orange 3, Disperse violet 1, Disperse blue 1 and mixtures thereof; and hair swelling agents including, but not limited to, urea, thiourea, acetic acid, phosphoric acid, formic acid, formamide, ethyl amine, alkali halides such as potassium iodide, sodium bromide, lithium bromide, and lithium chloride, and mixtures thereof.

High Solvent Containing Liquid Crystals

As will be evident to one skilled in the art, both the water-miscible solvents and the aqueous composition will both typically have low viscosity and therefore, as expected, the mixture of the two compositions will have low viscosity. Low viscosity compositions are not generally accepted in consumer products since they are difficult to handle and can lead to difficulty in evenly distributing the compositions onto the substrate which is to be treated. This uneven distribution results in uneven treatment of the substrate.

Thickening of aqueous compositions are well known in the art, however little is known to thicken solvents, like the water-miscible solvents of the present invention. Surprisingly, it has been discovered that lamellar liquid crystalline bi-layers can be formed and maintained within a predominantly solvent environment to provide viscosity build over a time period in excess of that required to deliver the solvent composition to treated substrate. Therefore, compositions comprising relatively high levels of water-miscible solvent can be thickened, an thereby made much more consumer acceptable, by these lamellar liquid crystals.

These high-solvent compositions comprise greater than 25%, preferably ranging from about 25% to about 98%, more preferably from about 50% to about90% of water-miscible solvent, from about 0.5% to about 25%, preferably from about 1.0% to about 10%, more preferably from about 2.0% to about 5% of surfactant, and water wherein the high-solvent composition is thickened by high solvent containing lamellar liquid crystals. The high solvent containing liquid crystals of the present invention can be in the form of flat sheets, unilamellar vesicles and multilamellar vesicles or combinations thereof.

Amphiphilic molecules, such as surfactants, are poorly soluble in water, their critical micellar concentration is between 10⁻⁸ and 10⁻¹² M (depending on their hydrocarbon chain length). At higher concentrations the amphiphiles form aggregates. Such aggregates vary in topology and stability depending on the structure of the amphiphilic molecule and surrounding conditions. The most common forms of amphiphile aggregates are micelles, cubic, hexagonal and lamellar phases. [See Polish Journal of Pharmacology, 1999, 51, 211-222 ISSN 1230-6002].

It is well known that in aqueous solutions, certain amphiphilic molecules and combinations form lamellar liquid crystals (neat phase) which are mesomorphic structure types between liquids, which are random, and crystals, which are periodic in three dimension. [See F. B. Rosevear, “Liquid Crystals: The Mesomorphic Phases of Surfactant Compositions”, J. Soc. Cosmetic Chemists, 19, 581-594 (Aug. 19, 1968)]. Such lamellar liquid crystals consist of parallel bimolecular layers, termed bi-layers, swollen in water wherein the hydrophilic polar head groups orient toward the aqueous environment and the hydrophobic tail regions orient toward the center where they are shielded from the aqueous environments. Parallel sheets of water separate successive bi-layers to create a multi-bi-layered system.

If desired, such lamellar liquid crystalline bi-layers can be dispersed via processing to form vesicles (also termed liposomes) wherein the bi-layers adopt a spherical configuration to surround an aqueous space. Liposomes consisting of a single bi-layer are referred to as unilamellar vesicles (ULV), and those consisting of two or more bi-layers (onion-like structures) as multilamellar vesicles (MLV). ULV's can be small (<100 nm in diameter) or large (>100 ; nm). MLV's can be 100 nm to >1 mm in diameter.

Many amphiphiles, or surfactants, are known to form lamellar liquid crystals in water depending on the degree of packing of adjacent surfactants within aqueous solution. This degree of packing is influenced by surfactant structure as well as packing synergisms that may exist between differing surfactants within mixed surfactant systems. As a general rule of thumb, the Critical Packing Factor (φ) can be utilized to visualize the surfactant structural characteristics that influence the degree of packing:

φ=v/(a×L)

where v is the volume occupied by hydrophobic groups within the core, a is the cross-sectional area occupied by the hydrophilic groups at the surface, and L is the length of the hydrophobic groups. For instance, as the hydrophobic group becomes bulkier, the packing will be increased. Also, it can be visualized that the addition of co-surfactants such as fatty alcohols (small a and large v and L) will also result in increased packing. While not being bound to theory, it is generally believed that the packing parameter (φ) needs to be greater than or equal to about 0.5 and less than or equal to about 1.0 for bi-layers to be formed. If φ<0.5 the formation of micelles will be favorable and if φ>1.0 the formation of reverse micelles will be favorable.

Suitable surfactants for forming the high solvent containing liquid crystalline bi-layer structures of this invention include cationic, anionic, nonionic, and gemini surfactants.

Suitable nonionic surfactants are fatty alcohols or fatty acids, or derivatives thereof, or a mixture of any of these, having a chain length of from about 14 to about 22 carbon atoms. These materials may be predominantly linear or may be branched. Some examples include myristyl alcohol, myristic acid, cetyl alcohol, palmitic acid, cestearyl alcohol, stearyl alcohol, stearic acid, oleic acid, oleyl alcohol, arachidyl alcohol, arachidic acid, behenyl alcohol, behenic acid, and mixtures thereof.

Other non-ionic surfactants suitable for use in the compositions of the present invention include condensation products of aliphatic (C16 to C22) primary or secondary linear or branched chain alcohols or phenols with alkylene oxides, usually ethylene oxide, and generally having from 1 to 30 ethylene oxide groups. Some examples include, but are not limited to, ceteth-1, ceteth-2, ceteth-3, ceteth-4, ceteth-5, ceteth-6, ceteth-10, ceteth-12, ceteth-14, ceteth-15, ceteth-16, ceteth-20, ceteth-24, ceteth-25, ceteth-30, ceteareth-2, ceteareth-3, ceteareth-4, ceteareth-5, ceteareth-6, ceteareth-7, ceteareth-8, ceteareth-9, ceteareth-10, ceteareth-11, ceteareth-12, ceteareth-13, ceteareth-14, ceteareth-5, ceteareth-16, ceteareth-17, ceteareth-18, ceteareth-20, ceteareth-22, ceteareth-23, ceteareth-24, ceteareth-25, ceteareth-27, ceteareth-28, ceteareth-29, ceteareth-30, steareth-2, steareth-3, steareth-4, steareth-5, steareth-6, steareth-7, steareth-8, steareth-30, steareth-11, steareth-13, steareth-14, steareth-15, steareth-16, steareth-20, steareth-21, steareth-25, steareth-27, steareth-30, arachideth-20, beheneth-5, beheneth-10, beheneth-20, beheneth-25, beheneth-30 and mixtures thereof.

Other suitable non-ionic surfactants include mono- or di-alkyl alkanolamides with carbon chain lengths of between 12 and 22 carbon atoms.

Long chain (C12-22) esters of polyols and sugars, the polyethoxylated and/or polypropoxylated alkylphenols, the polyhydroxylated polyethers of fatty alcohols, fatty acid alkanolamides, amine oxides, and the condensation products of ethylene oxide with long chain amides are also representative of suitable nonionic surfactants.

Examples of suitable cationic surfactants include quaternary ammonium halides, e.g., alkyltrimethylammonium hydroxides in which the alkyl group has from about 12 to 22 carbon atoms, for example dodecyltrimethyl-ammonium chloride, hexadecyltrimethylammonium chloride, cetyltrimethylammonium chloride, behenyltrimethylammonium chloride, benzyltrimethylammonium chloride, octyldimethylbenzyl-ammonium chloride, decetyldimethylbenzylammonium chloride, stearyldimethylbenzylammonium chloride, distearyldimethylammonium chloride, didodecyldimethylammonium chloride, dioctadecyldimethylammonium chloride, tallow trirnethylammonium chloride, cocotrimethylammonium chloride, cetylpyridinium chloride and their other corresponding halide salts and hydroxides. Preferred cationic surfactants are cetyltrimethylammonium chloride (CTAC) and cetyltrimethylamrnmonium bromide (CTAB). CTAB 99% from Fluka, CTAC 50% (Arquad 16-50, Akzo). Preferably, cationic surfactants are used at 2-10% with CTAC and CTAB being the preferred cationic surfactants. Additionally, when cationic surfactants are used, it is preferred to also employ cholesterol wherein the ratio of cholesterol to cationic surfactant ranges from 0.1:1.0 to 1.0:1.0, more preferably from 0.5:1.0 to 1.5:1.0, and most preferably 0.7:1.0 to 1.25:1.0.

Suitable anionic surfactants are the alkyl sulfonates, alkyl ether sulfonates, alkylaryl sulfonates, alkanoyl isethionates, alkyl succinates, alkyl sulfosuccinates, N-alkoyl sarcosinates, alkyl phosphates, alkyl ether phosphates, alkyl ether carboxylates, and alpha-olefin sulfonates, especially their sodium, magnesium, ammonium and mono-, di- and triethanolamine salts. The alkyl and acyl groups generally contain from 14 to 22 carbon atoms and may be unsaturated. The alkyl ether sulfates, alkyl ether phosphates and alkyl ether carboxylates may contain from one to ethylene oxide or propylene oxide units per molecule. A preferred anionic surfactant includes cetearyl phosphate.

Gemini surfactants are made up of two long hydrocarbon chains (C₁₂-C₂₂) and two ionic head groups linked by a short spacer. The spacer is attached directly to the identical ionic groups, each of which is in turn bonded to an identical hydrocarbon chain. The spacer can vary in length, hydrophobicity and flexibility and is typical a C₂-C₅ divalent alkyl radical. A typical gemini surfactant is as follows:

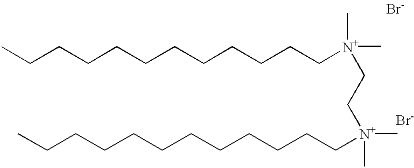

Gemini surfactants are described further in the book: Surfactants and Polymers in Aqueous Solution, by Bo Jonsson, Bjorn Lindman, Krister Holmberg and Bengt Kronberg, pages 4-5, John Wiley and Sons, ©1998.

The liquid crystals may also be formed from lipid surfactants including either phospholipids, i.e., based on glycerol and sphingosine, or glycolipid, i.e. based on sphingosine. Phospholipids are preferred with phosphatidyl choline (lecithin) being the preferred phospholipid. Of the alcohol moieties which comprise the phosphoglycerides, serine, choline and ethanolamine are particularly preferred, and of the fatty chains, those having a chain length of C14 to C24 are preferred. The fatty acid chains may be branched or unbranched, saturated or unsaturated, and palmitic, myristic, oleic, stearic, arachidonic, linolenic, linoleic and arachidic acids are particularly preferred.

Preferred surfactants for use in the present invention are of the nonionic type and include C16-20 fatty alcohols, and C16-20 fatty alcohol ethoxylates with 1 to 30 ethylene oxide groups. Specific examples include cetearyl alchol, cetyl alcohol, stearyl alcohol, arachidyl alcohol, oleyl alcohol, ceteareth ethoxylates with between 10 and 30 ethylene oxide groups, ceteth ethoxylates with between 10 to 30 ethylene oxide groups, steareth ethoxylates with between 10 and 30 ethoxylates, and combinations thereof. Preferably, C16-20 fatty alcohols are used in combination with C16-20 fatty alchol ethoxylates at a ratio of between 10:1 to 0.5:1, more preferably between 6:1 and 1:1, and most preferably between 5:1 and 1.5:1.

The surfactant(s) may be present in the composition in a total amount of from about 0.5 to 25% by weight, preferably from 1.0 to 10% by weight, more preferably from 1.5 to 6% by weight.

The lamellar liquid crystals may be comprised of one or more differing surfactants or lipid surfactants of the same or differing classes. The lamellar liquid crystals may be in the form of closed vesicles that may be unilamellar or multilamellar or in the form of flat sheets or combinations thereof. When spherical vesicles are formed, their size can be from 0.1 to 10 microns.

The presence of lamellar liquid crystals can be detected with the use of a polarizing microscope as is taught in “The Microscopy of the Liquid Crystalline Neat and Middle Phases of Soaps and Synthetic Detergents”, by F. B. Rosevear in The Journal of the American Oil Chemists' Society, Vol. 31, (December 1954).

Numerous techniques can be employed to produce the lamellar liquid crystals. In general these techniques involve mixing the components that form the liquid crystals, as well as any components that are to be incorporated within the bi-layers, under conditions that permit the formation of the liquid crystals.

The specific method for producing the liquid crystals is not critical. For instance, the lamellar liquid crystals can be pre-formed within the aqueous phase by incorporating the surfactants within the aqueous composition or they can be formed in-situ during product usage by incorporating the surfactants within the anhydrous composition.

For the former, the liquid crystals are pre-formed in the aqueous compartment by heating the surfactants within the aqueous phase to 60C accompanied by simple mixing or high shear mixing. Generally, a portion of the solvent is included within the aqueous compartment to facilitate a more rapid viscosity build upon intermixing with the solvent. While not being bound to theory, it is believed that the liquid crystalline bi-layers formed within the aqueous compartment are initially disrupted upon intermixing with the anhydrous solvent, after which they reform in the predominantly solvent environment. By including a portion of the solvent within the aqueous compartment, it has been found that the initial viscosity disruption is minimized with the resulting viscosity build happening considerably faster, i.e., within seconds.

For in-situ lamellar liquid crystal formation during product usage, the surfactants are pre-solubilized within the anhydrous solvent composition. This results in a non-structured solution that is thin in rheology. However, it has surprisingly been discovered that upon introduction of even small levels of water (<25 wt. %), the surfactants spontaneously form very structured lamellar liquid crystals within seconds. Thus, upon inter-mixing the thin anhydrous composition comprising the surfactant with the separate thin aqueous composition, lamellar liquid crystalline thickening is induced in-situ that facilitates product application to the hair.

Product Form

The aqueous and anhydrous compositions according to the present invention may be provided in any suitable physical form provided that they can be amply intermixed together by the consumer prior or simultaneous to application to hair, for example as low to moderate viscosity liquids. These compositions may be produced by procedures well known to the skilled artisan. The cosmetic compositions can be used in various manners as other known compositions in the art including but not limited to various rinse-off and leave-on applications such as hair shampoos, skin cleansers, skin lotions, hair conditioners, hair dyes, hair permanent waves, hair straighteners, hair bleaches, styling sprays, hair mousses, two-in-one shampoos, fabric softeners, lotions, nail polishes, hair serums, hair dyes, hair waving, etc.

The aqueous and anhydrous compositions should be stored in a manner that separates the two for over the shelf life of the product while providing ample protection to the atmosphere/environment. This can be accomplished via employment of a two package or two compartment execution wherein one package or compartment contains the anhydrous composition and the other package or compartment contains the separate aqueous composition. A number of mechanisms can be used to ensure that the two compositions are mixed either immediately prior or during use by the consumer including, but not limited to,

a dual or multi-chamber package whereby the anhydrous compositon is stored within one chamber and the aqueous compositon within the other chamber and wherein the contents of all the chambers are intermixed via or after dispensing for use, e.g., dispensing through the same orifice, dispensing through separate orifices followed by mixing within one's hands, dispensing through separate orifices followed by mixing upon application to hair or skin, or combinations thereof

multiple packages wherein the anhydrous composition is stored within one of the packages, e.g., bottle, vial, sachet etc., and the separate aqueous composition is stored within a separate package, e.g., bottle, vial or sachet, and whereby the volume of one of the packages is sufficiently large enough to enable the addition of the contents of the remaining packages, via pouring, dispensing etc., to enable intermixing of each of the separate compositions prior to use,

pouring the contents of both packages or compartments into one's hands which can be mixed by rubbing the hands together or via massaging onto the keratin substrate, and

a multichambered package whereby the anhydrous composition is stored within one chamber and the aqueous composition within the other chamber and wherein the chambers are separated by temporary ‘barriers’ that are removed or compromised prior to use to enable mixing, e.g., deformable barrier(s), breakable of barrier(s), removable barrier(s) etc Conversely, either of the anhydrous or aqueous compositions can be placed within shear sensitive (or other induced release) capsules which are dispersed within the other respective composition and which rupture (or other induced release) prior or during use.

### METHOD OF USE

The treatment composition of the present invention can be applied to wet, partially wet or dry substrate in need of treatment. Preferred embodiment of the treatment compositions can be applied to wet hair, partially wet hair or dry hair. The anhydrous composition can be applied directly to wet hair or the anhydrous composition and separate aqueous composition are intermixed (via packaging dispensing, pouring and shaking, hand rubbing, massaging onto substrate etc.) and applied to the hair with the hands, which may be gloved, and massaged in thoroughly. The contact time between the cosmetic composition of the present invention and the substrate can vary between 10 seconds and about 1 hour, preferably between 20 seconds and 30 minutes, more preferably between 30 seconds and 15 minutes. The composition is then thoroughly rinsed from the hair, though the composition can be applied as a leave-on product.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention. Ingredients are identified by chemical or CTFA name, or otherwise defined below.

All percentages herein are based upon the total weight of the compositions, and all such weight percentages as they pertain to listed ingredients are based on the active level and, therefore, do not include carriers or by-products that may be included in commercially available materials.

### EXAMPLE 1

### A Non-Limiting Example of an Anhydrous Dual Compartment System

| Aqueous Compartment | | Anhydrous Compartment | |
|---|---|---|---|
| ethanolamine⁶ | 0.38% | Polymer 1¹ | 0.75% |
| acetic acid⁷ | 0.25% | 1-methyl-2-pyrrolidone (NMP)² | 45.25% |
| perfume⁸ | 1.00% | cetyl alcohol³ | 1.50% |
| benzyl alcohol⁹ | 0.50% | stearyl alcohol⁴ | 1.50% |
| phenoxyethanol¹⁰ | 0.30% | ceteareth 25⁵ | 1.00% |
| tetrasodium EDTA¹¹ | 0.10% | | |
| Acusol OP301¹² | 2.00% | | |
| 1-methyl-2-pyrrolidone (NMP)² | 22.47% | | |
| DI water | 23.00% | | |
| Subtotal | 50.00% | Subtotal | 50.00% |
| | | Total | 100.00% |

¹Polymer 1 is as described herein, the preparation of which can be referenced in U.S. Ser. No. 09/478,855 by R. Glenn et. al.
²Available from Sigma-Aldrich Company Ltd, Dorset, England, 99.5%.
³Available as CO-1695 from Procter and Gamble, Cincinnati, Ohio.
⁴Available as CO-1895 from Procter and Gamble, Cincinnati, Ohio.
⁵Available as Volpo CS25 from Croda Oleochemicals, North Humberside, England
⁶Available from Sigma-Aldrich Company Ltd, Dorset, England, 99+%.
⁷Available from Sigma-Aldrich Company Ltd, Dorset, England, 99.7+%.
⁸Available from Procter and Gamble perfumers, Cincinnati, Ohio
⁹Available as Nipaguard MPA from Nipa-Hardwicke, Inc., Wilmington, Delaware.
¹⁰Available as Phenoxetol from Nipa-Hardwicke, Inc., Wilmington, Delaware.
¹¹Available as Cheelox 100 from Rhone-Poulenc, Cranbury, New Jersey.
¹²Available from Rohm and Haas, Philadelphia, Pennsylvania.

Example I can be prepared as follows:

Aqueous Compartment Composition

1. In a suitable container, add the water and the ethanolamine.

2. Adjust the pH down to 9.5 by adding approximately 0.25% Acetic Acid.

5. Continue mixing and add the benzyl alcohol, phenoxyethanol and the EDTA.

7. Add the 1-methyl-2-pyrrolidone.

8. Mix until homogenous

Anhydrous Compartment Composition

1. In a separate suitable container add the 1-methyl-2-pyrrolidone.

2. Heat the solution to 60° C. and add the ceteareth-25, cetyl alcohol & stearyl alcohol.

3. Mix with suitable agitation to combine but not to aerate for 30 minutes.

4. Cool batch at between 1-1.5° C./min to 30° C.

5. Add polymer 1 and agitate for approximately 4-6 hours while covered until polymer I dissolves.

6. Continue mixing and then add the Perfume.

The above two compositions are intermixed together prior or simultaneous to application to hair via mechanical action, e.g., dual dispensing package, mixing in hands, dual blister/sachet pack with removable barrier etc., and produce a resulting viscosity of greater than 10,000 cps that is comprised of lamellar liquid crystals.

### EXAMPLE II

### A Non-Limiting Example of an Anhydrous Dual Compartment System

| Aqueous Compartment | | Anhydrous Compartment | |
|---|---|---|---|
| cetyl alcohol³ | 1.50% | Polymer 2¹ | 0.75% |
| stearyl alcohol⁴ | 1.50% | 1-methyl-2-pyrrolidone (NMP)² | 49.25% |
| ceteareth 25⁵ | 1.00% | | |
| ethanolamine⁶ | 0.38% | | |
| acetic acid⁷ | 0.25% | | |
| perfume⁸ | 1.00% | | |
| benzyl alcohol⁹ | 0.50% | | |
| phenoxyethanol¹⁰ | 0.30% | | |
| tetrasodium EDTA¹¹ | 0.10% | | |
| Acusol OP301¹² | 2.00% | | |
| 1-methyl-2-pyrrolidone (NMP)² | 18.47% | | |
| DI water | 23.00% | | |
| Subtotal | 50.00% | Subtotal | 50.00% |
| | | Total | 100.00% |

¹Polymer 1 is as described herein, the preparation of which can be referenced in U.S. Ser. No. 09/478,855 by R. Glenn et. al.
²Available from Sigma-Aldrich Company Ltd, Dorset, England, 99.5%.
³Available as CO-1695 from Procter and Gamble, Cincinnati, Ohio.
⁴Available as CO-1895 from Procter and Gamble, Cincinnati, Ohio.
⁵Available as Volpo CS25 from Croda Oleochemicals, North Humberside, England
⁶Available from Sigma-Aldrich Company Ltd, Dorset, England, 99+%.
⁷Available from Sigma-Aldrich Company Ltd, Dorset, England, 99.7+%.
⁸Available from Procter and Gamble perfumers, Cincinnati, Ohio
⁹Available as Nipaguard MPA from Nipa-Hardwicke, Inc., Wilmington, Delaware.
¹⁰Available as Phenoxetol from Nipa-Hardwicke, Inc., Wilmington, Delaware.
¹¹Available as Cheelox 100 from Rhone-Poulenc, Cranbury, New Jersey.
¹²Available from Rohm and Haas, Philadelphia, Pennsylvania

Example II can be prepared as follows:

Aqueous Compartment Composition

1. In a suitable container, add the water and the ethanolamine.

2. Adjust pH down to 9.5 by adding approximately 0.25% Acetic Acid.

3. Heat solution to 65° C. and add ceteareth 25, cetyl alcohol & stearyl alcohol.

4. Mix with suitable agitation to combine but not to aerate for 30 minutes.

5. Continue mixing; add Perfume, Benzyl Alcohol, Phenoxyethanol and EDTA.

6. Cool batch at between 1-1.5° C./min to 30° C.

7. Add the 1-methyl-2-pyrrolidone.

8. Mix until homogenous

Anhydrous Compartment Composition

1. In a separate suitable container add Polymer I.

2. Add the 1-methyl-2-pyrrolidone and agitate for approximately 4-6 hours while covered until polymer I dissolves.

The above two compositions are intermixed together prior or simultaneous to application to hair via mechanical action, e.g., dual dispensing package, mixing in hands, dual blister/sachet pack with removable barrier etc., and produce a resulting viscosity of greater than 10,000 cps that is comprised of lamellar liquid crystals.

### EXAMPLE III

| Aqueous Compartment | | Anhydrous Compartment | |
|---|---|---|---|
| ethanolamine⁶ | 0.38% | Polymer 2¹ | 0.75% |
| acetic acid⁷ | 0.25% | 1-methyl-2-pyrrolidone (NMP)² | 45.25% |
| perfume⁸ | 1.00% | cetyl alcohol³ | 1.50% |
| benzyl alcohol⁹ | 0.50% | stearyl alcohol⁴ | 1.50% |
| phenoxyethanol¹⁰ | 0.30% | ceteareth 25⁵ | 1.00% |
| tetrasodium EDTA¹¹ | 0.10% | | |
| Acusol OP301¹² | 2.00% | | |
| 1-methyl-2-pyrrolidone (NMP)² | 22.47% | | |
| DI water | 23.00% | | |
| Subtotal | 50.00% | Subtotal | 50.00% |
| | | Total | 100.00% |

¹Polymer 2 is as described herein, the preparation of which can be referenced in U.S. Ser. No. 5,525,332 by A. D. Gough et. al.
²Available from Sigma-Aldrich Company Ltd, Dorset, England, 99.5%.
³Available as CO-1695 from Procter and Gamble, Cincinnati, Ohio.
⁴Available as CO-1895 from Procter and Gamble, Cincinnati, Ohio.
⁵Available as Volpo CS25 from Croda Oleochemicals, North Humberside, England
⁶Available from Sigma-Aldrich Company Ltd, Dorset, England, 99+%.
⁷Available from Sigma-Aldrich Company Ltd, Dorset, England, 99.7+%.
⁸Available from Procter and Gamble perfumers, Cincinnati, Ohio
⁹Available as Nipaguard MPA from Nipa-Hardwicke, Inc., Wilmington, Delaware.
¹⁰Available as Phenoxetol from Nipa-Hardwicke, Inc., Wilmington, Delaware.
¹¹Available as Cheelox 100 from Rhone-Poulenc, Cranbury, New Jersey.
¹²Available from Rohm and Haas, Philadelphia, Pennsylvania.

Example III can be prepared as follows:

Aqueous Compartment Composition

1. In a suitable container, add the water and the ethanolamine.

2. Adjust the pH down to 9.5 by adding approximately 0.25% Acetic Acid.

5. Continue mixing and add the benzyl alcohol, phenoxyethanol and the EDTA.

7. Add the l-methyl-2-pyrrolidone.

8. Mix until homogenous

Anhydrous Compartment Composition

1. In a separate suitable container add the 1-methyl-2-pyrrolidone.

2. Heat the solution to 60° C. and add the ceteareth-25, cetyl alcohol & stearyl alcohol.

3. Mix with suitable agitation to combine but not to aerate for 30 minutes. 4. Cool batch at between 1-1.5° C./min to 30° C.

5. Add polymer 1 and agitate for approximately 4-6 hours while covered until polymer I dissolves.

6. Continue mixing and then add the Perfume.

The above two compositions are intermixed together prior or simultaneous to application to hair via mechanical action, e.g., dual dispensing package, mixing in hands, dual blister/sachet pack with removable barrier etc., and produce a resulting viscosity of greater than 10,000 cps that is comprised of lamellar liquid crystals.

### EXAMPLE IV

### A Non-Limiting Example of an Anhydrous Dual Compartment System

| Aqueous Compartment | | Anhydrous Compartment | |
|---|---|---|---|
| ethanolamine⁶ | 0.38% | Conditioner 3¹ | 0.75% |
| acetic acid⁷ | 0.25% | 1-methyl-2-pyrrolidone (NMP)² | 45.25% |
| perfume⁸ | 1.00% | cetyl alcohol³ | 1.50% |
| benzyl alcohol⁹ | 0.50% | stearyl alcohol⁴ | 1.50% |
| phenoxyethanol¹⁰ | 0.30% | ceteareth 25⁵ | 1.00% |
| tetrasodium EDTA¹¹ | 0.10% | | |
| Acusol OP301¹² | 2.00% | | |
| 1-methyl-2-pyrrolidone (NMP)² | 22.47% | | |
| DI water | 23.00% | | |
| Subtotal | 50.00% | Subtotal | 50.00% |
| | | Total | 100.00% |

¹Conditioner 3 is as described herein, the preparation of which can be referenced in U.S. Ser. No. 5,087,733 by T. M. Deppert et. al.
²Available from Sigma-Aldrich Company Ltd, Dorset, England, 99.5%.
³Available as CO-1695 from Procter and Gamble, Cincinnati, Ohio.
⁴Available as CO-1895 from Procter and Gamble, Cincinnati, Ohio.
⁵Available as Volpo CS25 from Croda Oleochemicals, North Humberside, England
⁶Available from Sigma-Aldrich Company Ltd, Dorset, England, 99+%.
⁷Available from Sigma-Aldrich Company Ltd, Dorset, England, 99.7+%.
⁸Available from Procter and Gamble perfumers, Cincinnati, Ohio
⁹Available as Nipaguard MPA from Nipa-Hardwicke, Inc., Wilmington, Delaware.
¹⁰Available as Phenoxetol from Nipa-Hardwicke, Inc., Wilmington, Delaware.
¹¹Available as Cheelox 100 from Rhone-Poulenc, Cranbury, New Jersey.
¹²Available from Rohm and Haas, Philadelphia, Pennsylvania.

Example IV can be prepared as follows:

Aqueous Compartment Composition

1. In a suitable container, add the water and the ethanolamine.

2. Adjust the pH down to 9.5 by adding approximately 0.25% Acetic Acid.

5. Continue mixing and add the benzyl alcohol, phenoxyethanol and the EDTA.

7. Add the 1-methyl-2-pyrrolidone.

8. Mix until homogenous

Anhydrous Compartment Composition

1. In a separate suitable container add the 1-methyl-2-pyrrolidone.

2. Heat the solution to 60° C. and add the ceteareth-25, cetyl alcohol & stearyl alcohol.

3. Mix with suitable agitation to combine but not to aerate for 30 minutes.

4. Cool batch at between 1-1.5° C./min to 30° C.

5. Add polymer 1 and agitate for approximately 4-6 hours while covered until polymer I dissolves.

6. Continue mixing and then add the Perfume.

The above two compositions are intermixed together prior or simultaneous to application to hair via mechanical action, e.g., dual dispensing package, mixing in hands, dual blister/sachet pack with removable barrier etc., and produce a resulting viscosity of greater than 10,000 cps that is comprised of lamellar liquid crystals.

What is claimed is:

1. An anhydrous treatment composition comprising:
a) a reactive agent,
b) a solvent in which the reactive agent is soluble and which is water-miscible.

2. An anhydrous treatment composition according to claim 1 which comprises from about 0.01% to about 10%, by weight of the composition, of the reactive agent, and from about 25% to about 95%, by weight of the composition, of the water-miscible solvent.

3. An anhydrous treatment composition according to claim 2, wherein the reactive agent is selected from the group consisting of electrophilic groups, nucleophilic groups or protected thiol groups.

4. An anhydrous treatment composition according to claim 3, wherein the reactive agent additionally comprises a cosmetically active functional group.

5. A anhydrous treatment composition according to claim 4, wherein the reactive agent comprises an electrophilic reactive group selected from the group consisting of halotriazines, haloquinoxalines, halopyrimidines, vinylsulfones, β-haloethylsulfones, β-sulfatoethylsulfones, acrylates, methacrylates, acrylamides, methacrylamides, maleimides, epoxides, acylhalides, esters, carbamates, dithiocarboxylic acid esters, alkoxysilanes, thiosulfates, anhydrides, urea derivatives, isothiocyanates, isocyanates, lactones, thiosulfates, isothiuroniums, azalactones and mixtures thereof.

6. An anhydrous treatment composition according to claim 4, wherein the reactive agent comprises a nucleophilic reactive group selected from the group consisting of thiols, thiolates, thiols or thiolates containing quaternary salts, thioalkyl esters, thioalkylamides, thiol or thiolate derivatives of cysteamine, and mixtures thereof.

7. An anhydrous treatment composition according to claim 4, wherein the reactive agent comprises a protected thiol reactive group having the formula
R—(S—Pr)ₘ
where R is a mono or multivalent cosmetically active functional group, S is sulfur, Pr is a protecting group and m is an integer between 1 and 100.

8. An anhydrous treatment composition according to claim 7, wherein the protecting group is selected from the group consisting of heterocyclic protecting groups, Sp aliphatic trigonal carbon protecting groups, sp³ carbon electrophilic protecting groups, phosphorus protecting groups, metal based protecting groups, non-metal and metalloid based protecting groups other than phosphorus, energy-sensitive protecting groups and mixtures thereof.

9. An anhydrous treatment composition according to claim 1, wherein the water-miscible solvent has a Vaughan Solubility Parameter of from about 8.0 (cal/cm³)^{0.5} to about 17.0 (cal/cm³)^{0.5}.

10. An anhydrous treatment composition according to claim 1, wherein the water-miscible solvent is selected from the group consisting of ethyl formate, dimethyl isosorbide, acetylacetone, 2-butanone, acetone, methyl acetate, ethyl acetate, propyl acetate, ethoxyethanol, dipropylene glycol monomethyl ether, butyl lactate, t-butyl alcohol, phenyl acetate, 2-propoxyethanol, 2-isopropoxyethanol, methoxypropanol, isopropyl lactate, hexyl alcohol, butoxyethanol, tripropylene glycol (PPG-3), triacetin, methoxyethanol, isopropyl alcohol, PEG-8, methyl lactate, PEG-6, PEG-5, PEG-4, N-methylpyrrolidone, propyl alcohol, dipropylene glycol (PPG-2), acetonitrile, phenoxyethanol, triethylene glycol, hexylene glycol, ethyl alcohol, y-butyrolactone, butylene glycol, propylene carbonate, dimethyl sulfoxide, diethylene glycol, ethoxydiglycol, propylene glycol, pyrrolidone, pyrrolidone-2, methyl alcohol, ethylene carbonate, ethylene glycol, acetamide, glycerin, butyl carbitol, 1,3-dioxolane, dimethoxymethane, 1,2-hexanediol, dipropylene glycol butyl ether, dipropylene glycol t-butyl ether, propionaldehyde, diethoxymethane, glycerol formal, γ-valerolactone, α-methyl-γ-butyrolactone, and butyl acetate, ethanol, isopropyl alcohol, and mixtures thereof.

10. An anhydrous treatment according to claim 9, wherein the water-miscible solvent is selected from the group consisting of N-methylpyrrolidinone, propylene carbonate, γ-butyrolactone, γ-valerolactone, propylene glycol, dipropylene glycol, ethoxydiglycol, ethoxyethanol, dimethoxymethane, dimethyl isosorbide, butyl lactate, and mixtures thereof.

11. A composition comprising
a) greater than about 25% water-miscible solvent,
b) from about 0.5% to about 25% surfactant, and
c) water
wherein the composition is thickened by high solvent containing lamellar liquid crystals.

12. A composition according to claim 11 wherein the surfactant has a Critical Packing Factor greater than about 0.5 and less than or equal to about 10.

13. A composition according to claim 12 wherein the surfactant is selected from the group consisting of fatty alcohols having a chain length of from about 14 to about 22; fatty acids having a chain length of from about 14 to about 22; condensation products of C16 to C22 aliphatic alcohols with alkylene oxides; mono- and di-alkyl alkanolamides with carbon chain lengths of from about 12 to about 22; long chain esters of polyols and sugars; polyethoxylated and/or polypropoxylated alkylphenols; polyhydroxylated polyethers of fatty alcolhols, fatty acid alkanolamides, and amine oxides; condenstaion products of ethylene oxide with long chain amids; quaternary ammonium halides in which the alkyl group has from about 12 to about 22 carbon groups; alkyl sulfonates, alkyl ether sulfonates, alkylaryl sulfonates, alkanoyl isethionates, alkyl succinates, alkyl sulfosuccinates, N-alkoyl sarcosinates, alkyl phosphates, alkyl ether phosphates, alkyl ether carboxylates, and alpha-olefin sulfonates; sodium, magnesium, ammonium and mono-, di- and triethanolamine salts of alkyl sulfonates, alkyl ether sulfonates, alkylaryl sulfonates, alkanoyl isethionates, alkyl succinates, alkyl sulfosuccinates, N-alkoyl sarcosinates, alkyl phosphates, alkyl ether phosphates, alkyl ether carboxylates, and alpha-olefin sulfonates, gemini surfactants; lipid surfactants; and mixtures thereof.

14. A composition according to claim 13 wherein the surfactant is selected from the group consisting of cetyltrimethylarnmonium chloride, cetyltrimethylammonium bromide, cetearyl phosphate, phosphatidyl choline, serine, choline, ethanolamine, palmitic acid, myristic acid, oleic acid, stearic acid, arachidonic acid, linolenic acid, linoeic acid and arachidic acid, cetearyl alcohol, cetyl alcohol, stearyl alcohol, arachidyl alcohol, oleyl alcohol, ceteareth ethoxylates with between 10 and ethylene oxide groups, ceteth ethoxylates with between 10 and 30 ethylene oxide groups, steareth ethoxylates with between 1- and 30 ethylates, and mixtures thereof.

15. A system for treating amino-acid based substrates comprising the anhydrous treatment composition of claim 1 and a separately packaged aqueous composition.

16. A system according to claim 15 wherein the aqueous composition further comprises a surfactant.

17. A system according to claim 15 wherein the anhydrous composition further comprises a surfactant.

18. A system according to claim 17 wherein the surfactant has a Critical Packing Factor greater than about 0.5 and less than or equal to about 10.

19. A system according to claim 18 wherein the surfactant is selected from the group consisting of fatty alcohols having a chain length of from about 14 to about 22; fatty acids having a chain length of from about 14 to about 22; condensation products of C16 to C22 aliphatic alcohols with alkylene oxides; mono- and di-alkyl alkanolamides with carbon chain lengths of from about 12 to about 22; long chain esters of polyols and sugars; polyethoxylated and/or polypropoxylated alkylphenols; polyhydroxylated polyethers of fatty alcolhols, fatty acid alkanolamides, and amine oxides; condenstaion products of ethylene oxide with long chain amids; quaternary ammonium halides in which the alkyl group has from about 12 to about 22 carbon groups; alkyl sulfonates, alkyl ether sulfonates, alkylaryl sulfonates, alkanoyl isethionates, alkyl succinates, alkyl sulfosuccinates, N-alkoyl sarcosinates, alkyl phosphates, alkyl ether phosphates, alkyl ether carboxylates, and alpha-olefin sulfonates; sodium, magnesium, ammonium and mono-, di- and triethanolamine salts of alkyl sulfonates, alkyl ether sulfonates, alkylaryl sulfonates, alkanoyl isethionates, alkyl succinates, alkyl sulfosuccinates, N-alkoyl sarcosinates, alkyl phosphates, alkyl ether phosphates, alkyl ether carboxylates, and alpha-olefin sulfonates, gemini surfactants; lipid surfactants; and mixtures thereof.

20. An article comprising:
a) a package having a first chamber and a second chamber, each chamber having a dispensing orifice,
b) an anhydrous treatment composition contained in the first chamber, and
c) an aqueous composition contained in the second chamber.

21. A method for treating amino-acid based substrates comprising dampening of the amino-acid based substrate with water and applying a safe and effective amount of the treatment composition of claim 1 to the substrate.

22. A method for treating hair comprising dampening of the hair with water and applying a safe and effective amount of the treatment composition of claim 1 to the substrate.

23. A method for treating amino-acid based substrates comprising the mixing of the anhydrous treatment composition of claim 1 with water and applying a safe and effective amount of the composition-water mixture to the substrate.

24. A method for treating hair comprising the mixing of the anhydrous treatment composition of claim 1 with water and applying a safe and effective amount of the composition-water mixture to the hair.

25. A method for treating amino-acid based substrates comprising the mixing of the anhydrous treatment composition of claim 1 with water and applying a safe and effective amount of the composition-water mixture to the substrate.

26. A method for treating hair comprising the mixing of the anhydrous treatment composition of claim 1 with water and applying a safe and effective amount of the composition-water mixture to the hair.

27. A method for treating amino-acid based substrates comprising the simultaneous dispensing the anhydrous treatment composition and aqueous composition of the system of claim 15, mixing the compositions, and applying the resulting mixture to hair

28. A method for treating hair comprising the simultaneous dispensing the anhydrous treatment composition and aqueous composition of the system of claim 15, mixing the compositions, and applying the resulting mixture to hair

29. A method of thickening compositions containing greater than 25% water-miscible solvent comprising adding water and adding a solvent to form high solvent containing lamellar liquid crystals.
